(19) United States
(12) Patent Application Publication
(10) Pub. No.: US 2016/0243051 A1
(43) Pub. Date: Aug. 25, 2016

(54) MONO DISPERSE POLYMER NANOPARTICLES, FUNCTIONALIZED NANOPARTICLES AND CONTROLLED FORMATION METHOD

(71) Applicant: THE CURATORS OF THE UNIVERSITY OF MISSOURI, Columbia, MO (US)

(72) Inventors: Srikar RAMAN, Columbia, MO (US); Raghuraman KANNAN, Columbia, MO (US); Jatin VIMAL, Tamil Nadu (IN)

(73) Assignee: The Curators of the University of Missouri, Columbia, MO (US)

(21) Appl. No.: 15/030,755

(22) PCT Filed: Oct. 24, 2014
(86) PCT No.: PCT/US2014/062234

§ 371 Date: Apr. 20, 2016

Related U.S. Application Data
(60) Provisional application No. 61/895,100, filed on Oct. 24, 2013.

(51) Int. Cl. A61K 9/51; B01J 2/04; C08F 120/14; A61J 3/02

(52) U.S. Cl. CPC A61K 9/5192; A61J 3/02; B01J 2/04; A61K 9/5138; C08F 120/14

(57) ABSTRACT

A method produces polymer nanoparticles. Polymer solution is sprayed through a nozzle toward a collector. An electric field is created at the nozzle, such as by a voltage is applied to the nozzle to create the electric field. The voltage applied to the nozzle is from ˜10 (Kilovolt) to ˜30 (Kilovolt), distance from nozzle tip to collector is from ˜1 (centimeter) to ˜10 (centimeter) and the polymer concentration from ˜0.01% to ˜0.5% w/w. Preferably a grounded liquid collectors is used. The invention provides biocompatible monodisperse polymer nanoparticles having a size of less than ˜300 nm, preferably less than ˜150 nm. Payloads can be associated, and maintain efficacy, including more than one payload such as therapeutic agents and diagnostic agents on the same particles. Preferred particles are poly(methyl methacrylate) (PMMA-COOH) or acrylate analogues.

### PRIORITY CLAIM AND REFERENCE TO RELATED APPLICATION

The application claims priority under 35 U.S.C. §119 and from all applicable statutes and treaties from prior provisional application Ser. No. 61/895,100 which was filed Oct. 24, 2013.

### FIELD

A field of the invention is nano materials. The present invention concerns polymeric nanoparticles, formation methods, and application. Example applications include imaging, diagnostics and drug delivery.

### BACKGROUND

Polymeric nanoparticles of biodegradable and biocompatible polymers are of interest for various applications, and are of particular interest for controlled drug delivery and drug targeting. A variety of techniques have been researched for forming the polymeric nanoparticles and for functionalizing the nanoparticles to carry payloads such as drugs. Example formation techniques for creating drug delivery polymeric nanoparticles include bulk mixing, high pressure homogenization, nanoprecipitation and double emulsion. Kumaresh S. Soppimath, Tejraj M. Aminabhavi, Anandrao R. Kulkarni, Walter E. Rudzinsk, “Biodegradable polymeric nanoparticles as drug delivery devices,” Journal of Controlled Release, Volume 70, Issues 1-2, Pages 1-20, (2001).

Nano precipitation and double emulsion techniques emulsify an organic solvent with the polymer in oil in water (O/W) to obtain polymeric nanoparticulate compounds. Modifications of the basic processes are obtained via solvent effects, concentration effects, high pressure homogenization, and similar variations. The common synthetic procedure remains as an oil in water or water in oil emulsification process. Problems arise due to the toxicity caused due to the presence of residual organic solvents and residual monomers. Catarina Pinto Reis, Ronald J. Neufeld, Antonio J. Ribeiro, Francisco Veiga, “Nanoencapsulation I. Methods for preparation of drug-loaded polymeric nanoparticles,” Nanomedicine: Nanotechnology, Biology and Medicine, Volume 2, Issue 1, Pages 8-21 (2006).

Synthesis of PMMA (poly(methyl methacrylate)) nanoparticles using MMA (methyl methacrylate) monomer through an emulsification process has also been reported. A. N. Mendes, I. Hubber, M. Siqueira, G. M. Barbosa, D. L. Moreira, C. Holandino, J. C. Pinto, M. Nele, “Preparation and Cytotoxicity of Poly (Methyl Methacrylate) Nanoparticles for Drug Encapsulation,” Macromolecular Symposia, 319, 34-40 (2012). A significant limitation of this approach is toxicity due to the presence of MMA monomers and residual organic solvents, which leads to low drug encapsulation efficiency. The emulsion techniques also produce undesirably higher sized (larger than 100 nm) polydispersed particles. In addition, the particles produced by these techniques have a strong tendency toward aggregation. Some reported efforts to address these problems limit the polymer concentration to 0.1% weight of polymer/volume of solvent. However, decreasing polymer concentration leads to additional challenges with respect to drug encapsulation.

Drug delivery systems (DDS) should remain in systemic circulation for a predetermined period of time, generally tens of hours, for effective delivery of encapsulated compounds. Systemically administered DDS nanoparticles should remain in circulation for a longer time to increase their accumulation in targeted tissues before being cleared by the reticuloendothelial system, and be effectively internalized within the targeted cells. The accumulation can be influenced significantly by the physicochemical characteristics of nanoparticles, such as particle size, surface properties, and particle shape. Particles or molecules substantially larger than about 300 nm and polydisperse particles tend to ineffectively and insufficiently internalize within vasculatures. See, Yuan, Fan; Dellian, Marc; Fukumura, Dai; Leunig, Michael; Berk, David A.; Torchilin, Vladimir P.; Jain, Rakesh K., Cancer Research “Vascular permeability in a human tumor xenograft: molecular size dependence and cutoff size,” 55 (17), 3752-6 (1995). This defeats efficacy for systematic drug delivery for large particles as drug delivery.

Other prior techniques produce PMMA nanoparticles through co-polymerization of PEG with MMA prior to polymerization for forming nanoparticles. See, e.g. Jin Soak Kim and Ji Ho Youk, “Encapsulation of nanomaterials within intermediary layer cross-linked micelles using a photo-cross-linking agent,” Macromolecular Research, vol. 17, issue 11, pp 926-30 (2009). This approach can result in the nanoparticles having PEG present on both surface as well as within the nanoparticles. This presence may lead to drawbacks such as i) huge variations in the drug release properties since PEG is highly soluble in water, ii) unprecedented degradation of the nanoparticles, and (iii) changes in the intrinsic physiochemical properties of PMMA and its analogues.

Javorek published a review of more than 40 works concerning synthesis of particles using Electrohydrodynamic (EHD) processes. The processes only demonstrated producing particles of 300 nm or more. A. Jaworek, “Micro- and nanoparticle production by electrospraying,” Powder Technology, Volume 176, Issue 1, Pages 18-35 (2007).

### SUMMARY OF THE INVENTION

A method produces polymer nanoparticles. Polymer solution is sprayed through a nozzle toward a collector. An electric field is created at the nozzle, such as by a voltage is applied to the nozzle to create the electric field. The voltage applied to the nozzle is from ˜10 (Kilovolt) to ˜30 (Kilovolt), distance from nozzle tip to collector is from ˜1 (centimeter) to ˜10 (centimeter) and the polymer concentration from ˜0.01% to ˜0.5% w/w. Preferably a grounded liquid collectors is used. The invention provides biocompatible monodisperse polymer nanoparticles having a size of less than ˜300 nm, preferably less than ˜150 nm. Payloads can be associated, and maintain efficacy, including more than one payload such as therapeutic agents and diagnostic agents on the same particles. Preferred particles are poly(methyl methacrylate) (PMMA-COOH) acrylate analogues.

### LIST OF ACRONYMS

DDS drug delivery system

EHD electrohydrodynamic

ICG indocyanine green

MMA methyl methacrylate

PEG polyethylene glycol

PMMA poly(methyl methacrylate).

### BRIEF DESCRIPTION OF THE DRAWINGS

FIG. 1 is a schematic diagram that illustrates a preferred embodiment electrospray method for synthesis of PMMA-COOH nanoparticles (nanoparticles).

FIG. 2 is a schematic diagram of a preferred embodiment dual compound encapsulated nanoparticles;

FIG. 3A is schematic diagram illustrating a preferred synthesis method for the surface functionalization of nanoparticles with PEG;

FIG. 3B is schematic diagram illustrating stage-wise a preferred process for surface functionalization;

FIGS. 4A and 4B are TEM images of experimentally produced ICG encapsulated nanoparticles of the invention;

FIG. 5 is a data graph of size distribution results (DLS results) of experimentally synthesized non-Pegylated and Pegylated nanoparticles with encapsulated ICG;

FIG. 6 is a data graph that shows UV-Vis spectroscopy of an ICG solution and ICC encapsulated nanoparticles solution of the invention that was produced in experiments;

FIG. 7 is a graph illustrating stability analysis of experimentally produced nanoparticles of the invention with respect to size based on DLS measurements; error bars represent the standard deviation from the average.

FIGS. 8A-8C are images of whole body imaging florescent imaging of mice using IVIS imaging systems illustrating retention of experimentally produced nanoparticles;

FIG. 9 is a graph of data illustrating quantitative analyses detailing the normalized mean intensity for whole body imaging of mice with experimentally produced nanoparticles.

### DESCRIPTION OF THE PREFERRED EMBODIMENTS

The present invention relates to the development of novel and stable polymeric nanoparticles for application in imaging, diagnostics and drug delivery systems. Preferred methods of the invention provide a composition controlled high field electrohydrodynamic formation method. The method can produce polymeric nanoparticles and nanoparticle drug delivery systems. The nanoparticles can be monodisperse and small, ˜300 nm or less, and preferably ˜150 nm or less.

Preferred nanoparticles can carry more than one payload. Multiple compounds, such as multiple drugs, can be associated with preferred embodiment nanoparticles.

A preferred controlled electrospray method of the invention can integrate multiple compounds into one polymeric matrix, wherein the desired intrinsic properties and integrity of the polymeric matrix of any given compound present in the matrix are not adversely affected by the presence of the polymeric matrix or the presence of any other encapsulated compound or group of compounds. These attributes give independent and associative properties to each individual component forming the nanoparticulate system.

Preferred embodiments include surface functionalized nanoparticles. Embodiments provide carboxyl functionalized nanoparticles.

Particular preferred formation methods of the invention form poly (methyl methacrylate-co-methacrylic acid) (PMMA-co-MAA also called as PMMA-COOH) and its other acrylate analogues through a high field electrohydrodynamic method that provides control over nanoparticle size and provides monodisperse particles. Preferred embodiments provide PMMA-COOH nanoparticles that are monodisperse and ˜300 nm or less. Particular preferred embodiments are monodisperse and 150 nm or less. Particular sizes can be selected by adjusting formation parameters and can be selected to depend upon the payload(s) to be carried. More than one payload can be incorporated to form a drug delivery vehicle during the formation method.

In preferred methods of the invention, the voltage for the electro spray is from ˜10 (Kilovolt) to ˜30 (Kilovolt), the distance from tip to collector is from ˜1 (centimeter) to ˜10 (centimeter) and polymer concentration from ˜0.01% to ˜0.5% w/w (±10%). In preferred embodiments, a liquid collector is used, and preferably grounded de-ionized water. Preferred embodiments of the invention provide highly uniform nanoparticles made up of PMMA-COOH and its analogues. The methods of the invention advantageously allow excellent control of particle size. Uniform mondisperse particles of small size provide reduced clearance from a patient by the reticuloendothelial system, and more effective internalization by targeted cells.

Other preferred embodiments include analogues of PMMA-COOH, that within the context of the present methods, include polymers that have a molecular weight at least and preferably greater than 5 kDa and preferably greater than 25 kDa and sufficient viscoelasticity to transform into nanoparticles. Particular preferred analogues of PMMA-COON include polyethyl acrylate), Poly(butylacrylate), poly(methyl acrylate), copolymers of neutral, alkaline and acidic ethyl acrylate and methyl acrylate polymers, Ammonio Methacrylate Copolymers, Aminoalkyl Methacrylate Copolymers, copolymers of vinyllactams such as poly(methyl methacrylate) PMMA, poly (2-hydroxyethyl methacrylate) PHEMA, and poly[N-(2-hydroxypropyl)methacrylamide. Preferably, the polymer is selected from poly(methyl methacrylate) PMMA-COOH and its analogues.

Embodiments of the invention include mono disperse PMMA-COOH nanoparticles having a diameter of ˜300 nm or less, and in particular preferred embodiments ˜150 nm or less. Carried payloads can affect the size. Embodiments of the invention include an electrospray method for forming mono disperse PMMA-COOH nanoparticles.

While not necessary to patentability, and without being bound to the theory, embodiments of the invention are believed to the inventors to provide a first method for simultaneous reduction of the size of a DDS nanoparticle while also providing for greater control and homogeneity of the DDS nanoparticle. This invention provides methods for the preparation of homogenous DDS Nanoparticles. Another advantage of methods of the invention is an ability to utilized preformed polymer and co-polymers allowing “on-demand” surface functionalization based upon targeted application.

Unlike prior techniques mentioned in the background, preferred electrospray methods of the invention can integrate multiple compounds into one polymeric matrix. Desired intrinsic properties and integrity of the polymeric matrix and a compound present in the matrix are not adversely affected by the presence of the polymeric matrix or the presence of any other encapsulated compound or group of compounds. These attributes give independent and associative properties to each individual component forming the nanoparticulate system.

A preferred electrospray method generates monodispersed droplets. The droplet size can vary from tens of nanometers to hundreds of micrometers, depending on the processing parameters. The preferred process generates structured nanoparticles in a controlled manner and can provide high drug/nucleic acid encapsulation efficiency. The preferred methods avoid toxicity problems that are inherent to emulsification methods.

Particles of the invention can also be produced to have an affinity towards a specific cell or a surface modified to provide longer blood residence time. These features can provide enhanced permeability and retention, or cell specific targeting.

Preferred methods of the invention provide monodisperse nanoparticles of PMMA. PMMA-COOH or its analogues using preformed PMMA polymer or its analogues with relatively high encapsulation efficiency, drug loading efficiency and homogenous particle size distribution which has not been reported anywhere. Poly (methyl methacrylate-co-methacrylic acid) is a polymer made from co polymerization of Poly (methyl methacrylate) and methacrylic acid. The specific purpose of using this methacrylic component within PMMA is to provide the polymeric system with surface functionality. In other words, PMMA in presence of —COOH group are biocompatible as well as functionalizable through chemical conjugation.

Without being bound by the theory, embodiments of the invention are believed to provide a first method for encapsulation of multiple compounds within the PMMA nanoparticulate system for complementary functioning; for example, encapsulating a contrast agent as well as a therapeutic agent within each nanoparticle in the desired ratio to enable visualization along with therapy.

Experiments to demonstrate the invention have demonstrated the synthesis of nanoparticles of carboxyl functionalized PMMA nanoparticles using preformed Poly (methyl methacrylate-co-meth acrylic acid) polymer (PMMA-COOH).

Embodiments of the invention avoid problems such as associated with prior co-polymerization techniques discussed in the background that can lead to the intermittent formation of nanofibers along with the nanoparticles. Nanofibers are not desired for drug delivery applications. Nanoparticles of the invention can deliver payloads in vivo. Advantageously, preferred embodiment PMMA-COOH nanoparticles are biocompatible but are not biodegradable. In a diagnostic or therapeutic method of the invention, PMMA-COOH nanoparticles are delivered in vivo and allowed to collect in a quantity at a targeted location with an associated payload. Associated payloads can be encapsulated, embedded, attached, conjugated, or impregnated on or in the nanoparticle, or adsorbed at its surface. In preferred methods of in vivo release, the payload is release in response to applied external energy, such as ultrasound or electromagnetic energy. In other preferred methods, the payload is released via enzymatic, protein or chemical reaction.

Molecule(s) or compound(s) can be associated as payloads without any chemical modification of the molecule or the compound for directly or indirectly assisting therapeutic or diagnostic effect. Molecules can be attached to the surface via covalent bonds, electrostatic bonds, or physically adsorption, including Van der Waal forces (conjugation).

Surface functionalization agents including zero-length crosslinkers, i.e. carbodiimides and its derivatives particularly EDC (carbodiimides), are engaged in this invention. They cause direct conjugation of carboxylates (—COOH) to primary amines (—NH2) without becoming part of the final crosslink (amide bond) between target molecules. Further, NHS ester i.e. N-Hydroxysuccinimide were utilized for further surface functionalization steps with reactive groups formed by EDC-activation of carboxylate molecules.

Preferred polymer nanoparticles can deliver associated therapeutic agents as payloads through systemic, oral, buccal, sublingual, ocular, topical, transdermal, nasal, pulmonoary and/or rectal administration to a patient.

Preferred embodiment polymer nanoparticles can have single or multiple compounds associated as a payload. In preferred embodiments, single or multiple compounds are encapsulated or embedded within each nanoparticle. The integrity and properties of the compound and the nanoparticles are maintained during formation.

Preferred payloads can be bioactive agents selected from a group that includes an antiproliferative agent, an anti-inflammatory agent, an antineoplastic, are antimitotic, an antiplatelet, an anticoagulant, an antifibrin, an antithrombin, a cytostatic agent, an antibiotic, an anti-allergic agent, an anti-enzymatic agent, an angiogenic agent, a cyto-protective agent, central nervous systems agents, antibacterials, a cardioprotective agent, and an antioxidant or any combination thereof.

Additional preferred payloads can be one or more active pharmaceutical ingredients embedded or encapsulated within the nanoparticles, and can also include also contrast agents.

Preferred nanoparticles include an active agent carried by the particle, such as a drug, a contrasting agent and combinations of same, embedded, conjugated, impregnated, or encapsulated in the nanoparticle, or adsorbed at the surface of the nanoparticle.

Preferred embodiments provide a method of preparing nanoparticles of Pegylated PMMA-COOH and its analogues. The method includes activation of the carboxyl group of PMMA-COOH and its analogues with Carbodiimides and NHS Esters, and chemical conjugation of PEG onto the surface of the nanoparticles through NH+-COO-linkage between PEG and the nanoparticles.

Preferred imaging methods of the invention include particle enhanced X-ray/Computed tomography (CT) or Magnetic Resonance Imaging (MRI). Embodiments provide a method of diagnosis in a subject's body a target cell or target tissue. Nanoparticles of the invention include a contrast agent and are associated with one or more targeting agents effective to target delivery to a target cell or target tissue.

Preferred embodiment polymer nanoparticles are associated with multiple compounds and provide for complementary functioning. Complementary function can include, for example, a contrast agent as well as a therapeutic agent that can be associated with each nanoparticle in a predetermined desired ratio to provide both imaging enhancement and therapy.

Preferred polymer nanoparticles are both non biodegradable and biocompatible and have a surface sterically stabilized with hydrophilic molecules. Fabrication methods of the invention provide the ability for surface modifications, steric stabilization, surface functionalization, and general characteristic tailoring to improve performance of nanoparticles in delivering therapeutic agents and diagnostic agents.

Many active ingredients can be associated with preferred nanoparticles. An active ingredient is a substance that, when administered to an organism, has a biological effect on that organism is considered to be active ingredient. Preferred nanoparticles can be associated with both hydrophilic and hydrophobic active ingredients.

Preferred nanoparticles can be associated with one or more bioactive agents selected from a group that includes an antiplatelet, an anticoagulant, an antifibrin, an antithrombin, a cytostatic agent, an antibiotic, an anti-allergic agent, an antiproliferative agent, an antiinflammatory agent, an antineoplastic, an antimitotic, an anti-enzymatic agent, an angiogenic agent, CNS drugs and Antibactierals, Antifungals, Local anesthetics, a cyto-protective agent, it cardioprotective agent, and an antioxidant or any combination thereof.

Preferred nanoparticles can be associated with pharmaceutically-active agents which may be employed in the present invention includes but not limited to drugs used for Alzheimer's disease, anesthetics, acromegaly agents, analgesics, antiasthmatics, anticancer agents, anticoagulants and antithrombotic agents, anticonvulsants, antidiabetics, antiemetics, antiglaucoma, antihistamines, anti-infective agents, antiparkinsons, antiplatelet agents, antirheumatic agents, antispasmodics and anticholinergic agents, antitussives, carbonic anhydrase inhibitors, cardiovascular agents, cholinesterase inhibitors, treatment of CNS disorders, CNS stimulants, contraceptives, cystic fibrosis management, dopamine receptor agonists, endometriosis management, erectile dysfunction therapy, fertility agents, gastrointestinal agents, immunomodulators and immunosuppressives, memory enhancers, migraine preparations, muscle relaxants, nucleoside analogues, osteoporosis management, parasympathomimetics, prostaglandins, psychotherapeutic agents, sedatives, hypnotics and tranquilizers, drugs used for skin ailments, steroids and hormones.

Preferred nanoparticles can be associated with Antineoplastics, such as Alkylating agents such as Nitrogen mustards, Cyclophosphamide, Mechlorethamine or Mustine (HN2), Uramustine or Uracil Mustard, Melphalan, Eniluracil, Chlorambucil, Ifosfamide, Bendamustine, Nitrosoureas, Carmustine, L-phenylalanine mustard, Lomustine, Streptozocin, Alkyl sulfonates such as Busulfan, Thiotepa, Procarbazine, Altretamine, Tetrazines (Dacarbazine, Mitozolomide, Temozotomide) and its analogues, Platinum-based chemotherapeutic drugs (termed platinum analogues) such as Picoplatin, Ormaplatin, Oxaplatin, Cisplatin, Carboplatin, Nedaplatin, Oxaliplatin, Satraplatin and Triplatin Tetranitrate, Antimetabolites such as Purine, Pyrimidine analogues, Antifolates, Base analogues, Nucleoside Analogues, Antinutrient such as (Azathioprine and Mercaptopurine), 5-azadeoxycytosine, Thioguanine, Fludarabine, Pentostatin, Cladribine, Arabinosylcytosine, Capecitabine, Gemcitabine and Decitabine, Pemetrexed, Mecaptopurine, Thioguanine Fludarabine phosphate, Fluorouracil, Floxuridine, Deoxycytidine, 5′-Deoxyflurouridine, 5-Azacytosine, Cytarabine, Capecitabine, Gemcitabine, Pentostatin, Methotrexate, Azathioprine, Camphothecin derivatives such as Camptothecin, 10-hydroxy-7-ethylcamptothecin (SN38), 9-Aminocamptothecin, 10,11-methylenedioxycamptothecin, Allopurinol, 2-chloroadenosine, Trimetrexate, 9-nitrocamptothecin, Amide derivatives such as Perfosfamide, Ifosphamide, Mefosphamide, Aminopterin derivatives such as Aminopterin, Methylene-10-Deazaaminopterin (MDAM), Epirubicin and karenitecin.

Preferred nanoparticles can be associated with Antineoplastic Antibiotics such as Antinomycins derivatives such as Dactinomycin, Anthracyclines derivatives such as Daunorubicin, Doxorubicin, Idarubicin, Aureolic acid derivatives such as Plicamycin, Mithramycin, Olivomycins, Chromomycins, variamycin, Bleomycin, Mithramycin, Mitomycin analogues such as Streptozocin, Acivicin, Calicheamicin, Plant products such as Vinka Alkaloids and their analogues such as Vincristine, Vinblastine, Vinrosidine, Vinleurosine, Vinglysinate, Vindesine, a Diterpene derivative or a Taxane such as Paclitaxel (or its derivatives such as DHA-Paclitaxel or PG-Paxlitaxel) or Docetaxel, Other Miscellaneous compounds like Irinotecan, Etoposide, Teniposide, Vinorelbine, Asparaginase, Pegaspargase, Altretamine, Mitoxantrone hydrochloride, Adriamycin, Gallium Nitrate, Arsenic trioxide, Bexarotene, Sargramostim, Filgrastim, Porfimer sodium, Mitotane, Leuprolide acetate, Triptoralen Pamoate, Goserelin acetate, Anastrozole, Letrozole and Exemestane, Interferons like Interferon Alfa-2a, Interferon Alfa-2b, Interferon Alfa-n3, Aldesleukin, Denileukin diftitox, Bacillus Calmette-Guerin (BG), Monoclonal antibodies like Rituximab, Gemtuzumb, Ozogamicin, Radiotherapeutic agents such as Chromic Phosphate P32, Sodium Phosphate P32, Sodium iodide 1 132, Strontium 89 Chloride, Samarium SM 153, Lexidronam, Cytoprotective agents such as MercaptoEthanesulfonic acid, Amifostine, Dexrazoxane, and Tromethamine, Amide derivatives such as Trifluoromethylaniline, Flutamide, Nilutamide, and Bicalutamide, Progesterone and its analogues such as Medroxyprogesterone, and Megesterol Acetate.

Preferred nanoparticles can be associated with Anti-inflammatory Analgesics, which includes Salicylic acid derivatives such as Sodium salicylate, Salicylamide, Asprin, Salsalate, Diflunisal, Sodium Thiosalicylate, Magnesium Salicylate, Choline Salicylate, Ammonium, Lithium, and Strontium Salts of salicylic acid, N-arylanthranitic acids derivatives including Mefenamic acid, Meclofenamate sodium, Arylacetic acid derivatives such as Indomethacin, Sulindac, Tolmetin Sodium, Ibuprofen, Naproxen, Dexibuprofen, Fenoprofen, Ketoprofen, Etodolac, Arylpropionic acid derivatives Oxaprozin, Piroxicam, Meloxicam, Cox-2 inhibitors such as Celecoxib, Rofecoxib and Valdecoxib, Aniline and p-Aminophenol derivatives such as Aniline, Acetanilid, P-Aminophenol, Formanilid, Benzanilid, Salicylanilide, Exalgin, Acetaminophen, Anisidine, Phenetidine, Phenacetin, Lactylphenetidin, Phenocoll, Kryofine, p-Acetoxy acetanilide, Phenetsal, Pertonal, Pyrazolone and Pyrazolidinedione Derivatives including Antipyrine, Aminopyrine, Dipyrone, Phenylbutazone, Oxyphenbutazone.

Preferred nanoparticles can be associated with Antiviral agents, which includes Nucleoside Antimetabolites such as Idoxuridine, uridine, Vidarabine, Acyclovir, Valacyclovir, Ganciclovir, Famciclovir and Penciclovir, Cidofovir, Foscarnet sodium, Reverse Transcriptase Inhibitors such as Zidovudine, Didanosine, Zalcitabine, Stavudine, Lamivudine, Micellaneous Nucleoside Antimetabolites like Rivavirin, Nonnucleoside Reverse Transcriptase inhibitors such as Nevirapine, Delavirdine, Efavirez, HIV protease inhibitors such as Saquinavir, Indinavir, Ritonavir, Amprenavir, and Nelfinavir.

Preferred nanoparticles can be associated with Antipsychotics, which includes Phenothiazines such as Promazine, Chlorpromazine hydrochloride, Triflupromazine Hydrochloride, Thioridazine Hydrochloride, Mesoridazine Besylate, Prochlorperazine Maleate, Perphenazine and Fluphenazine Hydrochloride, Ring analogues of Phenothiazines includes Thioxanthenes, Dibenzoxazepines, and Dibensodiazepines such as Thiothixene, Loxapine succinate and clozpine, Fluoro butyrophenones such as Haloperidol, Droperidol, Risperidone, Pimozide, Penfluridol, β-Aminoketones such as Molindone hydrochloride, Benzamides includes Remoxipride, Olanzapine and Quetiapine, Antimanic agents such as Lithium Salts such as Lithium carbonate, Lithium Citrate.

Preferred nanoparticles can be associated with Anticonvulsants or Antiepileptic Drugs, such as Barbiturates such as Mephobarbital, Hydantoins includes Phenytoin, Mephenytoin and Ethotoin, Oxazolidinediones such as Trimethadione, Succinimides includes Phensuximide, Methsuximide, Ethosuximide, Ureas and Monoacylureas includes Carbamazepine, Miscellaneous agents like Valproic acid, Gabapentin, Tiagabine, Felbamate, Lamotrigine, Zonisamide, Topiramate (Topamax), Benzodiazepines includes Clonazepam and Diazepam and Chloazepate etc.

Preferred nanoparticles can be associated with Antiarrhythmic agents, which includes such as Membrane Depressant Drugs such as Quinidine, Procainamide, Disopyramide, Lidocaine, Phenytoin sodium, Mexiletine, Tocainide, Flecainide Acetate, Moricizine, Propafenone, β-adrenergic Blocking agents such as Amiodarone, Bretylium Tosylate, Dofetilide, Ibutilide, Sotalol, Azimilide, Antiarrhythmics includes Verapamil, Diltiazem, Renin-Angiotensin system Inhibitors includes Lisinopril, ACE inhibitor Prodrugs includes Enalapril Maleate, Benazepril Hydrochloride, Quinapril Hydrochloride, Ramipril, Fosinopril sodium, Trandolapril, Angiotensin II blockers includes Losartan, Candesartan, Irbesartan, Valsartan, Adrenergic system Inhibitors includes Guanethidine derivatives such as Guanethidine Monosulfate, Guanadrel sulfate, Selective α-Adrenergic Antagonists includes Prazosin, Terazosin, Doxazosin, Centrally acting Adrenergic drugs includes Methyldopate, Clonidine, Guanabenz acetate, Guanfacine hydrochloride, Vasodilating agents includes Hydralazine, Sodium Nitroprusside, Potassium Channel Agonists includes Diazoxide, Minoxidil, Positive Inotropic agents such as Digoxin, Digitalis, Amrinone, Milrinone, Antihyperlipidemic agents such as Clofibrate, Gemfibrozil, Fenofibrate, Dextrothyroxine sodium, Colesevelam, HMG-CoA Reductase inhibitors includes Lovastatin, Simvastatin, Pravastatin, Fluvstatin and Atorvastatin and Cerivastatin, Anticoagulants includes Protamine sulfate, Dicumarol, Warfarin sodium, Anisindione, Hypoglycemic agents includes Sulfonylureas such as Tolbutamide, Chlorpropamide, Tolazamide, Acetohexamide, Glipizide, Glyburide, Glimepiride, Gliclazide, Nonsulfonyl ureas includes Repaglinide, Nateglinide, Thiazolindiones includes Rosiglitazone, Pioglitazone, Bisguanidines includes Metformin, α-Glucosidase inhibitors includes Acarbose Miglitol, etc.

Preferred nanoparticles can be associated with Antibiotics, which includes β-Lactam Antibiotics includes Penicillin G, Penicillin V, Nafcillin, Oxacillin, Cloxacillin, Dicloxacillin, Ampicillin, Amoxicillin, Cyclacillin, Carbenicillin, Ticarcillin, Piperacillin, Mezlocillin, Clavulanate Potassium USP, Sulbactam, Tazobactam, Carbapenems includes Thienamycin, Imipenem-Cilastatin, Meropenem, Biapenem, Cephalosporins includes Cephalexin, Cephradine, Cefadroxil, Cefachlor, Cefprozil, Loracarbef, Cefuroxime axetil, Cefixime, Cephalothin, Cephapirin, Cefazolin, Cefamandole, Cefonicid, Ceforamide, Cefuroxime, Cefotaxime, Ceftizoxime, Ceftriaxone, Ceftazidime, Cefoperazone, Cefoxitin, Cefotetan, Cefmetazole, Monobactams derivatives includes Stretomycin sulfate, Neomycin Sulfate, Paromomycin Sulfate, Kanamycin, Amikacin, Gentamicin sulfate, Netilmicin sulfate Sisomicin sulfate and Spectinomycin Hydrochloride, Tetracyclines derivatives includes Tetracycline, Rolitetracycline, Oxytetracycline hydrochloride, Chlortetracycline Hydrochloride, Methacycline Hydrochloride, Demeclocycline USP, Meclocycline sulfosalicylate, Doxycycline and Minocycline, Macrolides derivatives includes Erythromycin, Erythromycin Stearate, Erythromycin Ethylsuccinate, Erythromycin Estolate, Erythromycin Gluceptate, Erythromycin Lactobionate, Clarithromycin, Azithromycin, Dirithromycin, Troleandomycin, Lincomycins derivatives includes Lincomycin, Clindamycin Hydrochloride, palmitate and Phosphate, Polypeptide derivatives includes Vancomycin Hydrochloride, Teicoplanin, Bacitracin, Polymyxin sulfate B, Colistin Sulfate, Colistimethate sodium, Gramicidin, Chloramphenicol, Novobiocin sodium, Mupirocin, Quinupristin/Dalfopristin, Linezolid and Fosfomycin Tromethamine.

Preferred nanoparticles can be associated with diagnostic agents, which includes Technetium (^{99m}Tc), Fluorine (¹⁸F), Gallium (⁶⁷Ga), Iodine (¹³¹I), Indium (¹¹¹In), Oncoscint CR/OV, Thallium (²⁰¹T¹) and Xenon Compounds (¹³³Xe

Preferred nanoparticles provide biocompatibility in all its states—i.e., in its intact state, its synthesized state, and in its decomposed state i.e., its degradation products—to perform its desired function with respect to a medical therapy, without eliciting undue undesirable local or systemic effects in the recipient or beneficiary of that therapy.

Preferred nanoparticles can be administered in vivo via systemic and nonsystemic delivery and/or administration of nanoparticles to a subject. Administration can include, but is not limited to, injection, intravenous, subcutaneous, intramuscular, and intra depot formulations, oral, buccal, sublingual, transdermal, topical, ocular, nasal, pulmonary, and rectal formulations.

Preferred particular in vivo payload release methods including internal release trigger mechanism like cleavage of nanoconstruct through enzymatic, protein or other chemical action within physiological conditions and external trigger methods including Highly Focused ultrasound (HIFU), laser assisted ablation, or any other externally applied energy to trigger the drug delivery and diagnostic delivery in vivo.

Preferred nanoparticles can be associated with pharmaceutical composition payloads that can optionally contain other non-essential ingredients. For example, the composition can contain up to 10 weight percent of conventional pharmaceutical adjuvants. These adjuvants or additives include preservatives, stabilizers, antioxidants, pH adjusting agents, and viscosity modifying agents.

Particular example experiments and embodiments will now be discussed. Artisans will recognize broader aspects of the invention from the description of the experiments.

### EXPERIMENTS

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Materials

Poly (methylmethacrylate-co-eth acrylic acid) (PMMA-COOH, Mw 34 KDa), N-(3-dimethylaminopropyl)-N′-ethylcarbodiimide hydrochloride (EDC), N-hydroxysuccinimide (NHS), acetone, chloroform and indocyanine green (ICG) were obtained from Sigma-Aldrich (St. Louis, Mo.). Carboxyl-polyethylene glycol-amine terminated (cPEG-NH2, Mw 3 KDa) was purchased from Laysan Bio Inc. (Arab, Ala.). All materials were used without any further purification.

Synthesis of PMMA-COOH Nanoparticles

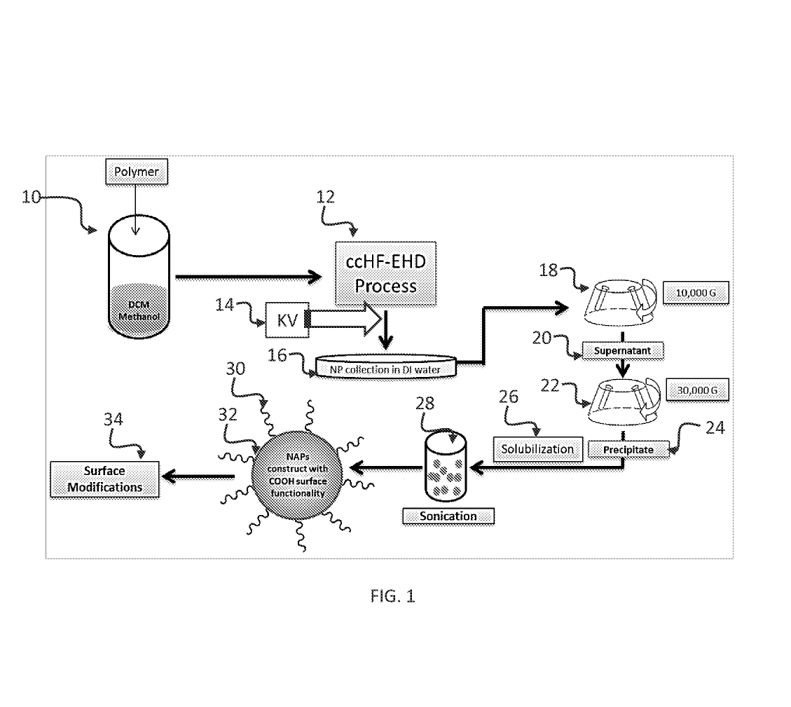

FIG. 1 illustrates the example preferred method. A polymer solution is prepared via normal techniques 10, such as in a DCM methanol. An electrohydrodynamic electrospray process 12 forms nanoparticles by driving the polymeric solution through a syringe pump at a constant flow rate. The present composition controlled high field electrohydrodynamic process method drove the polymeric solution through a syringe pump at a constant flow rate. The solution ejected through the tip of an insulated stainless-steel nozzle (gauge 21 in the experiments). A high positive potential field 14 is applied at the nozzle. A sufficient field is created by applying, for example, ˜10 (Kilovolt) to ˜30 (Kilovolt) to the nozzle. Similar fields can be created by less direct techniques, but the voltage application is direct and convenient. A preferred collector 16 is grounded de-ionized water. Using a liquid collector instead of a solid collector helps to avoid agglomeration of the particles due to the residual electrostatic forces present on the surface of the particles. Constant stirring of the collector is preferred to assists in producing a homogenous suspension of the nanoparticles. In an alternative process, the nanoparticles are collected onto a solid substrate and subsequently suspended the particles in aqueous media.

In the experiments, nanoparticles were electrosprayed using PMMA-COOH dissolved in Dichloromethane (DCM)/Methanol (Me) solvent. Different samples of nanoparticles included (1) 0.5% of PMMA-COOH and (2) 0.5% PMMA-COOH with encapsulated ICG (indocyanine green) were fabricated.

The experimental method was carried out at room temperature and ambient humidity. The parameters of the present composition controlled high field electrohydrodynamic experimental method are described in Table 1.

TABLE 1

| | PMMA- | | | | |
|---|---|---|---|---|---|
| | COOH | ICG | Solvent | Flow Rate | Voltage/TCD |
| S. NO. | (mg) | (mg) | (gm) | (ml/hr) | (KV/cm) |
| 1 | 30 | 0 | 6 | 2 | 2.85 |
| 2 | 30 | 10 | 6 | 2 | 2.85 |

Post-Processing

The nanoparticle suspension was then centrifuged 18 at 10,000 G for a period of 15 minutes to remove the small amount of larger particles (<150 nm). This produces a supernatant 18. The supernatant was then subjected to additional centrifuge 22 at 30,000 G for 1 hr to produce a precipitate 24. The precipitated pellet obtained from the centrifugation is solubilized 26 and then sonicated 28 for 15 mins. This produces monodisperse nanoparticles 30, which include a COOH surface functionality 32. Surface modifications 34 can then be performed, such as to associate a payload surface attachment.

Various adjustments can modify the size of the particles. The centrifugal speed is an example. It is possible to carry out size separation using various different media at various different speed and time. In addition, other forms of purification including filtration and subjecting to various gyration forces will also yield similar results.

Encapsulation

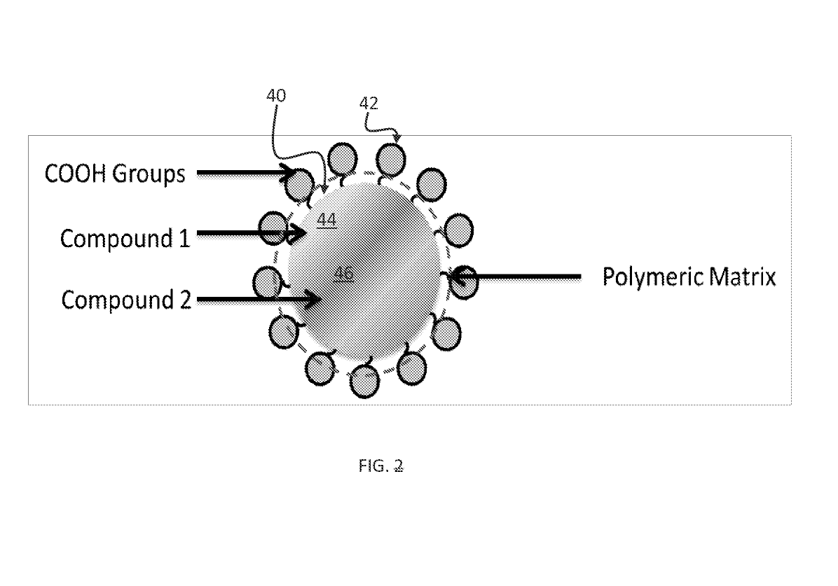

Encapsulation of therapeutic agents including drugs, proteins, and diagnostic agents including dyes and contrast agents was carried out experimentally. The agent(s) (individually or in combination), are dissolved or suspended homogenously in the solvent along with the polymers in step 10. Complete entrapment of molecules within nanoparticles during the experimental method was achieved. Payloads have different properties have been incorporated to demonstrate the robust nature of the present method, including two different payloads, such as therapeutic and contrast payloads. A nanoparticle 40 is represented in FIG. 2 with COOH groups 42 and first 44 and second 46 payload molecules.

Surface Modification—PEGylation

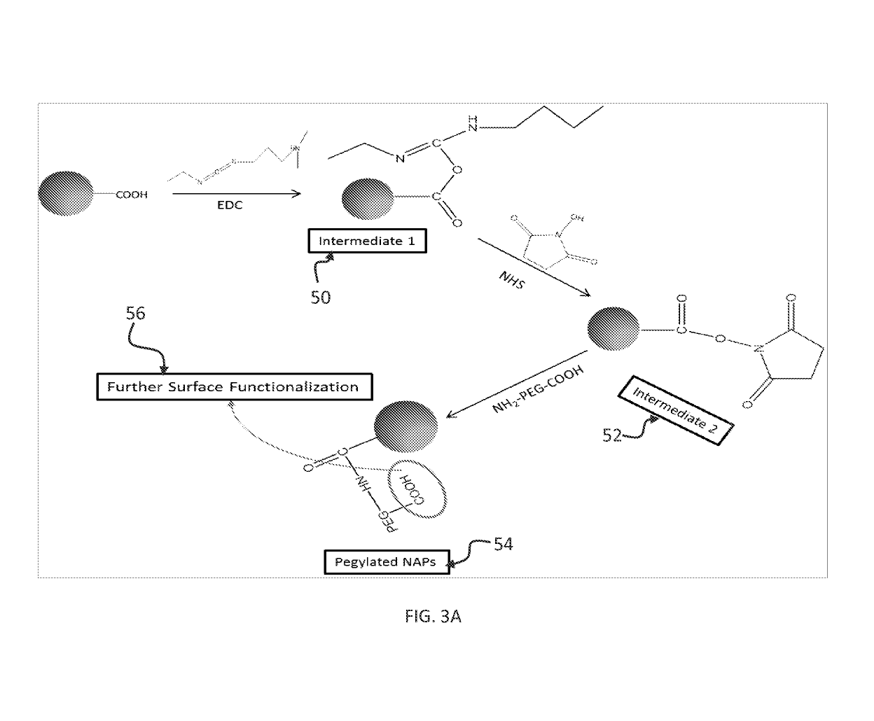

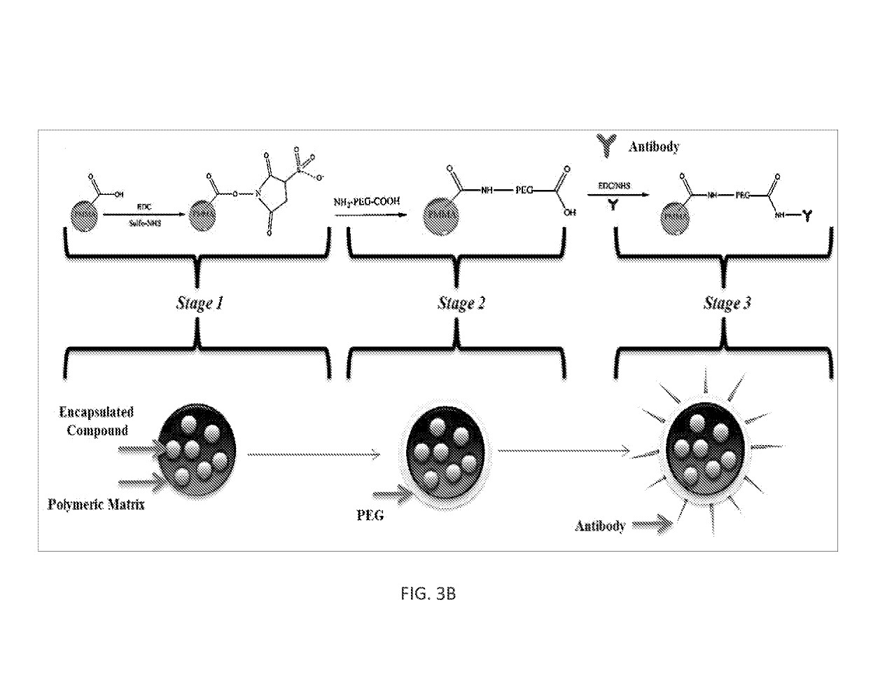

The inherent presence of carboxyl group (—COOH) present on the surface of the nanoparticles resultant from the formation technique and materials was utilized for the surface modification. FIG. 3A illustrates the process. Polyethylene glycol with one end group consisting of amine was conjugated to the carboxyl group of nanoparticles through EDC/NHS reaction. In brief, the nanoparticles pellet, obtained from the post-processing step, was suspended in 2-(N-morpholino)ethanesulfonic acid (2-(N-morpholino)ethanesulfonic acid (MES) buffer. The concentration of the nanoparticles in the buffer was approximately 1 mg/ml. 17 mg of EDC and 10 mg of NHS dissolved of MES buffer and was then added to the nanoparticles solution to produce an intermediate 50. The reaction was carried out for 3 hrs. Thereafter, the carboxyl activated nanoparticles were washed with MES and the excess EDC and NHS were removed to produce a second intermediate 52.

In order to conjugate PEG to the surface of the nanoparticles, the nanoparticles were re-suspended in PBS buffer and NH2-PEG-COOH was added in excess. Overnight reaction allowed the chemical conjugation of PEG onto the surface of the nanoparticles through NH+-COO-linkage between PEG and nanoparticles 54. The presence of COOH group present as end termination in PEG can further be utilized for antibody or ligand attachment in further functionalization 56. Alternatively, antibody and/or ligand could be functionalized to the surface of nanoparticles during the PEGylation step as a simultaneous reaction. FIG. 3B shows the alternative, where the encapsulated compound polymer particles are form in a first stage, the PEGylation is a second stage that includes antibody additional in a third stage.

Particle Size Characterization

The hydrodynamic size of the nanoparticles was characterized using dynamic light scattering method. Malvern Zetasizer Nano ZS was used for determining the particle size distribution. In addition, Transmission Electron Microscopy (TEM) was used to confirm the results from DLS measurements.

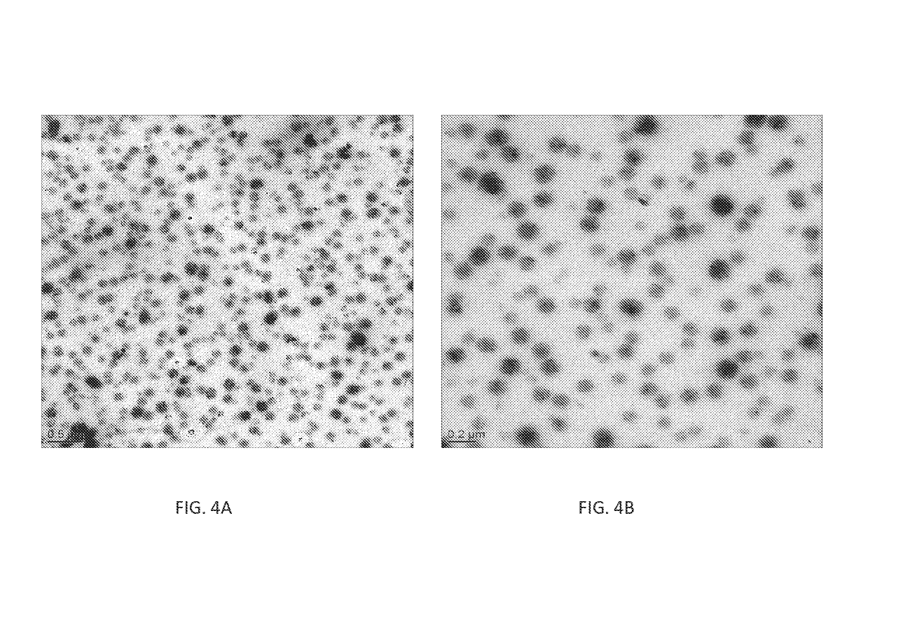

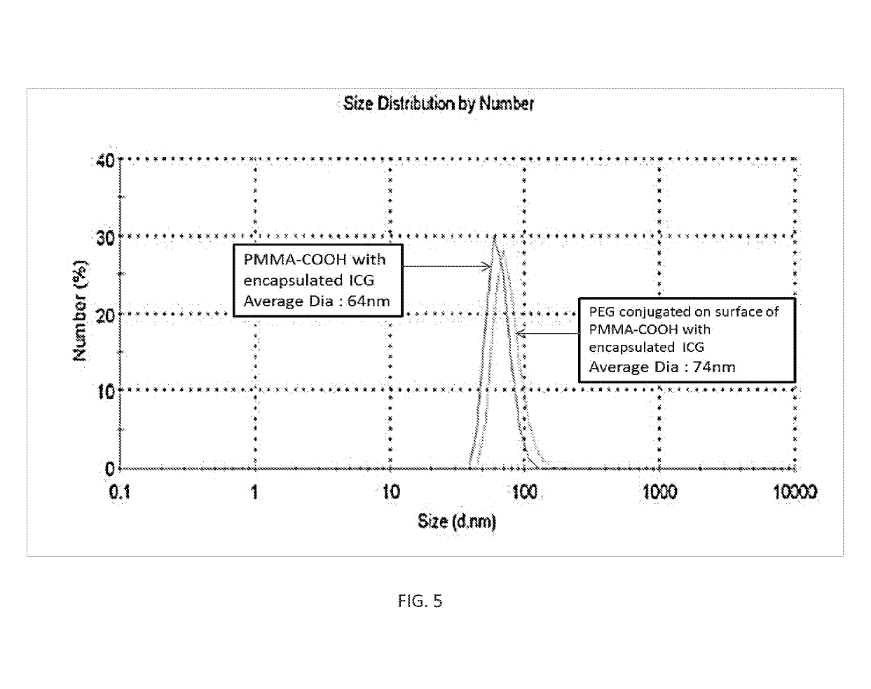

The size of the nanoparticles with encapsulated as well as pristine nanoparticles (with no encapsulated compound) is detailed in Table 2. Representative TEM images and Dynamic Light Scattering (DLS) results of the nanoparticles are shown in FIGS. 4A, 4B and 5. Various nanoparticles synthesized using the present method were found to be in the average size ranging from 50-90 nm, with low polydispersity index (PDI). The size of the nanoparticles increased by 1.5 nm after PEGylation, as shown in FIG. 5, indicating the presence of surface bound PEG molecules. In addition, no major change in the size was observed even with five-fold increased concentration of encapsulated ICG. TEM images confirm highly dispersed, non-aggregated, spherically shaped uniform nanoparticles.

TABLE 2

| | PMMA- | | | |
|---|---|---|---|---|
| | COOH (wt./wt of | ICG (wt./wt of | Encapsulated | Average |
| S. NO | solvent %) | polymer %) | compound | Size (nm) |
| 1 | 0.5 | 0 | ICG | 64 |
| 2 | 0.5 | 6.66 | ICG | 64 |
| 3 | 0.5 | 16.66 | ICG | 65 |
| 4 | 0.5 | 33.33 | ICG | 68 |

Loading Efficiency

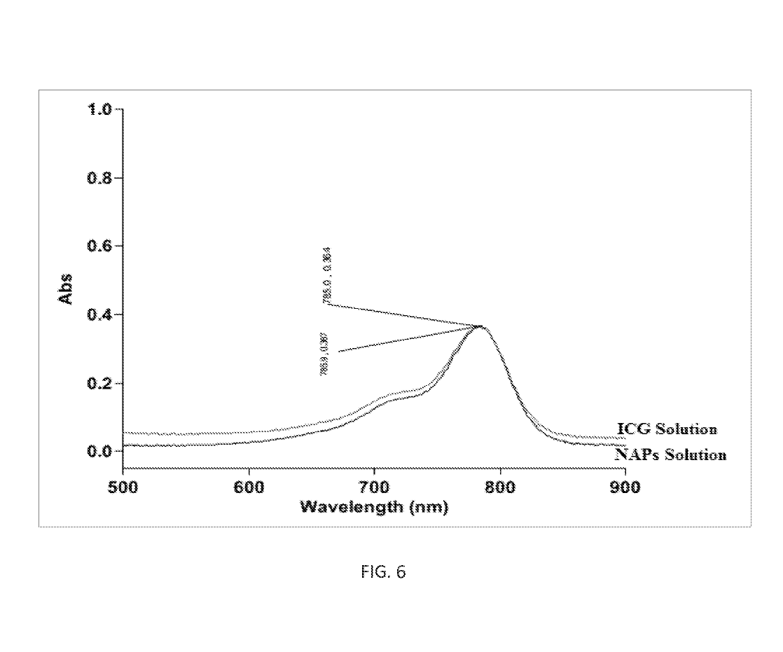

The loading efficiency of the encapsulated compound was computed UV-Vis-NIR spectroscopy. As an example, the amount of ICG present within PMMA-COOH was determined by dissolving the polymeric nanoparticles in methanol solution. The absolute value of absorption was then correlated to the calibration curve of ICG in methanol to determine the content of ICG present within the system. The % encapsulation was then calculated based on the following equation,

$$\% Encapsulation = \frac{{M}_{d}}{{M}_{d 0}} \times 100 ,$$

where, Md is the mass of the ICG determined using spectroscopic analysis, and Md0 is the initial dye loading during the start of the present composition controlled high field electrohydrodynamic method. FIG. 6 shows the UV-VIS spectroscopic analysis of ICG content present within the nanoparticles.

Nanoparticles synthesized through the present method with 10 mg ICG and 30 mg PMMA-COOH (initial content) were collected in 20 ml DI water. Theoretically, 1 mg of nanoparticles, if 100% encapsulation were to be obtained, would contain 750 μg of polymer and 250 μg of ICG. Based on calibration curve of ICG, it was determined that the amount of ICG present, after washing the surface bound ICG, to be approximately 165 mg. Thus, to confirm the amount of encapsulated compound, the solutions were suitable diluted to 10 μg/ml. The final sample for analysis consisted of 950 μl of methanol and 50 μl of DI water for both ICG and nanoparticles. It was evident from the analysis, as shown in FIG. 6, that equal amounts of ICG, namely, 165 μg, is present in both the sample solutions. Hence, the encapsulation efficiency was computed to be 65%.

Stability Studies

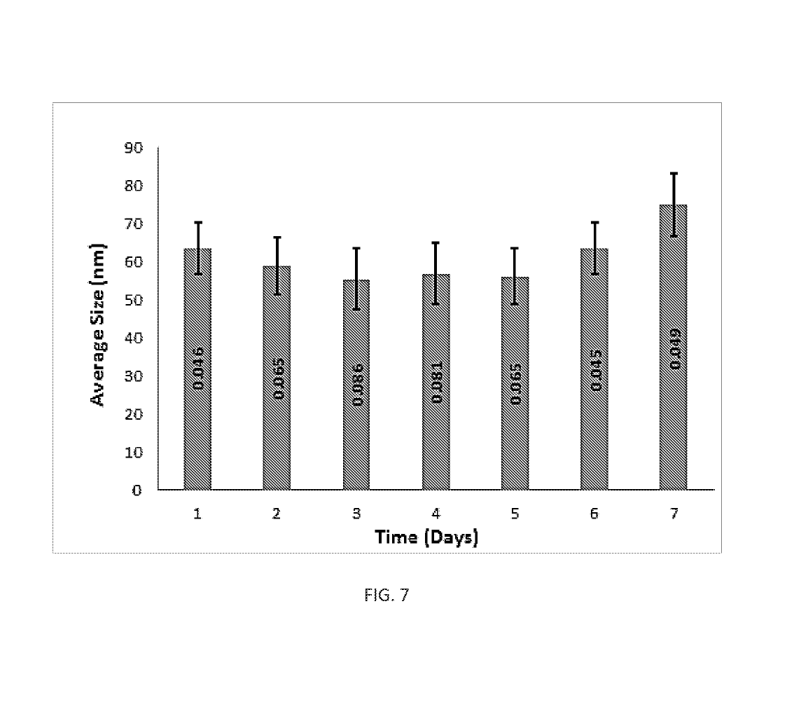

Stability of the nanoparticles was characterized by incubating the nanoparticles in DI water at 25° C. for a period of 7 days. The size and the zeta potential of the nanoparticles were investigated at period intervals of 24 hrs to determine if any form of degradation or aggregation had occurred. For this study 0.5% w/w polymer concentration was used in the present composition controlled high field electrohydrodynamic method and the nanoparticulate suspension was filtered through 0.2 micron Cellulose Acetate filters. The stability analysis of pristine nanoparticles in detailed FIG. 7.

It can be observed from the data in FIG. 6 that the Pristine PMMA-COOH nanoparticles are highly stable and experience almost no size range (mono disperse nanoparticles are obtained). The slight increase in the size of the nanoparticles suspended in DI water could be attributed to swelling of the nanoparticles. Lack of aggregation of the nanoparticles is shown due to (i) no significant size increase is observed, and (ii) the absolute value of below 0.2 remains almost constant as well.

In-Vivo Studies

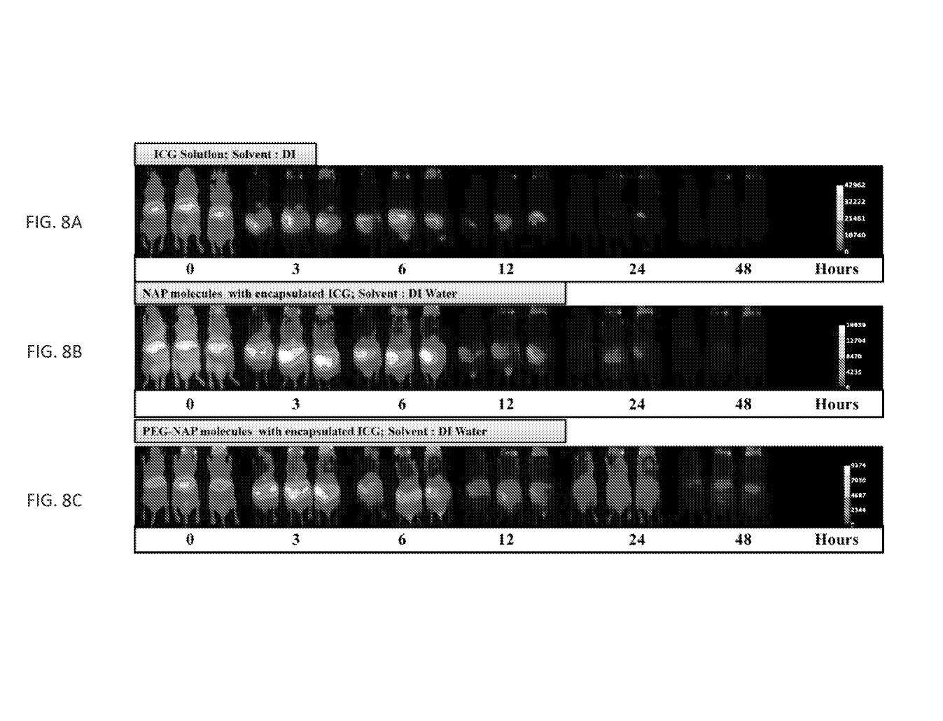

Near Infrared based florescent imaging was carried out in nude mice through IVIS in-vivo imaging system. Briefly, 100 μl of samples of free ICG solution (0.15 mg/ml), PMMA-COOH (0.15 mg/ml of ICG present in nanoparticles) and Pegylated nanoparticles containing ICG (0.15 mg/ml) were injected once and their florescence were recorded at various time points as shown in FIGS. 8A-8C. The results showed that the florescence of free diminished within 6 hrs and was completely reduced to almost zero within 24 hrs; whereas, the NIR florescence of PMMA-COOH was retained for 12 hrs before completely diminishing. On the other hand, Pegylated nanoparticles showed florescence for a longer period of time with low florescence present even after 48 hrs. There are three possible reasons for florescence quenching; namely, (i) degradation of free ICG physiological conditions, or (ii) rapid clearance of free ICG from the body, or (iii) a combination of both processes.

It can be inferred from FIG. 7 that Pegylated nanoparticles show higher florescence than the non Pegylated nanoparticles and free ICG solution, for the following reasons. Firstly, had there been a release of ICG from the nanoparticles, the florescence should have quenched within 6 hours, which did not occur, as observed in free ICG solution. Second, when nonPEG-nanoparticles is compared with ICG solution, the florescence was intact for a longer period of time for nanoparticles. Therefore, the following conclusions could be drawn by the above two observations: (i) ICG remained intact and encapsulated within the nanoparticles, and GO PEGylation of nanoparticles increased the blood circulation time.

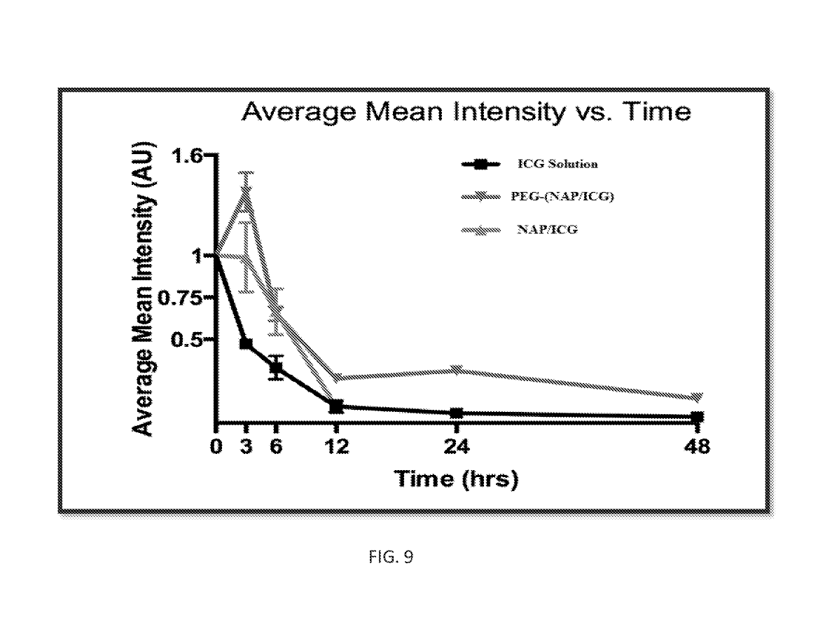

Quantitative analysis of mean intensity with respect to time confirmed the above observations as shown in FIG. 9. These results are a very positive indication for utilization of the candidate for selective and targeted release. A matrix which does not allow the leakage of the encapsulated molecule can be used for releasing the therapeutic agent at the desired locations. In other words, even if certain percentage of the nanoparticle complex does not target the desired locations, the therapeutic agent would not release and hence would not cause undesired harm to healthy cells.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A method for forming polymer nanoparticles, the method comprising:
spraying polymer solution through a nozzle toward a collector; and
applying an electric field around the nozzle while spraying, wherein a distance from nozzle tip to collector is from ˜1 (centimeter) to ˜10 (centimeter) and the polymer concentration from ˜0.01% to ˜0.5% w/w.

2. The method of claim 1, wherein the electric field is created by applying voltage to the nozzle; wherein the voltage is from ˜10 (Kilovolt) to ˜30 (Kilovolt).

3. The method of claim 1, wherein the collector comprises a liquid.

4. The method of claim 3, wherein the liquid comprises grounded de-ionized water.

5. The method of claim 1, wherein the polymer is a polymer comprising a polymer having a molecular weight of at least 5 kDa and sufficient viscoelasticity to transform into nanoparticles.

6. The method of claim 1, wherein the polymer comprises PMMA-COOH or acrylate analogues thereof.

7. The method of claim 1, wherein the acryl ate analogue is selected from poly(ethyl acrylate), Poly(butylacrylate), poly(methyl acrylate), copolymers of neutral, alkaline and acidic ethyl acrylate and methyl acrylate polymers, Ammonio Methacrylate Copolymers, Aminoalkyl Methacrylate Copolymers, copolymers of vinyllactams including poly(methyl methacrylate) PMMA, poly (2-hydroxyethyl methacrylate) PHEMA and poly[N-(2-hydroxypropyl)methacrylamide.

8. The method of claim 1, further comprising a preliminary step of preparing the polymer solution, wherein said preparing further comprises mixing a payload into the polymer solution, which payload becomes encapsulated during said spraying and applying.

9. The method of claim 8, wherein the payload comprises one of a therapeutic or diagnostic molecule.

10. The method of claim 9, wherein the payload comprises multiple therapeutic or diagnostic molecules.

11. The method of claim 10, wherein the payload comprises both a therapeutic and diagnostic molecule.

12. The method of claim 1, further comprising collecting and size separating nanoparticles to obtain uniformly sized monodisperse nanoparticles.

13. The method of claim 12, further comprising functionalizing the surface of the nanoparticles.

14. The method of claim 1, wherein the PMMA-COOH solution comprises a dichloromethane (DCM)/Methanol (Me) solvent.

15. The method of claim 1, wherein said spraying comprises flowing polymer at a steady rate through the nozzle.

16. The method of claim 1, further comprising encapsulating or embedding a payload within the nanoparticles.

17. The method of claim 16, wherein the payload comprises one of a pharmaceutically-active agent, anti-inflammatory agent, drug, or bioactive agent.

18. The method of claim 1, wherein the collector comprises a liquid and further comprising stirring the liquid during said spraying.

19. Biocompatible monodisperse polymer nanoparticles having a size of less than 300 nm.

20. The nanoparticles of claim 19 associated with a payload that is one of pharmaceutically-active agent, anti-inflammatory agent, or bioactive agent.

21. The nanoparticles of claim 19 embedding or encapsulating a payload that is a drug.

22. The nanoparticles of claim 19 embedding or encapsulating a plurality of payloads.

23. The nanoparticles of claim 19 wherein the polymer comprises poly(methyl methacrylate) (PMMA-COOH) or acrylate analogues.

24. The nanoparticles of claim 19 wherein the acrylate analogue is selected from poly(ethyl acrylate), Poly(butylacrylate), poly(methyl acrylate), copolymers of neutral, alkaline and acidic ethyl acrylate and methyl acrylate polymers, Ammonio Methacrylate Copolymers, Aminoallkyl Methacrylate Copolymers, copolymers of vinyllactams including poly(methyl methacrylate) PMMA, poly (2-hydroxyethyl methacrylate) PHEMA, and poly[N-(2-hydroxypropyl)methacrylamide.

25. The nanoparticles of claim 19 having a size of less than 150 nm.
